(19) United States
(12) Patent Application Publication
(10) Pub. No.: US 2014/0348770 A1
(43) Pub. Date: Nov. 27, 2014

(54) DEFORMATION MEANS FOR KERATIN-CONTAINING FIBERS CONVEYS STRONG HOLD AND NATURAL APPEARANCE

(71) Applicant: Henkel AG & Co., KGaA, Dusseldorf (DE)

(72) Inventors: Burkhard Mueller, Duesseldorf (DE); Susanne Schmarje, Hamburg (DE)

(73) Assignee: Henkel AG & Co. KGaA, Dusseldorf (DE)

(21) Appl. No.: 14/368,263

(22) PCT Filed: Nov. 9, 2012
(86) PCT No.: PCT/EP2012/072246

§ 371 Date: Jun. 23, 2014

(30) Foreign Application Priority Data
Dec. 22, 2011 (DE) 10 2011 089 578.7

(51) Int. Cl. A61K 8/81; A61Q 5/06

(52) U.S. Cl. CPC A61K 8/8152; A61K 8/8182; A61Q 5/06; A61K 2800/74
USPC 424/70.15

(57) ABSTRACT

Agents for treating keratin-containing fibers, in particular human hair, comprising a cosmetically acceptable carrier, and: (a) at least one strengthening anionic copolymer, comprising at least one structural unit of formula (I) and at least one structural unit of formula (II) and at least one structural unit of formula (III), and (b) at least one polymer comprising at least one structural unit of formula (M1), produce an excellent hold of a shape imposed on the fibers, e.g. a hairstyle. The keratin-containing fibers are given a pronounced shine and feel soft.

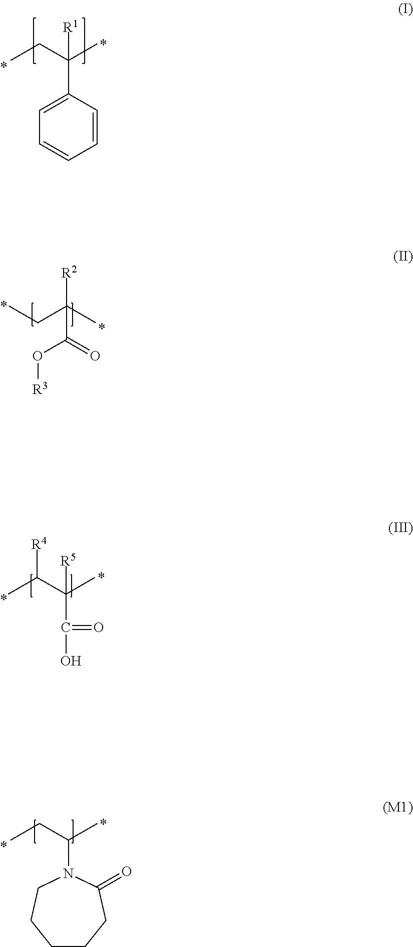

### CROSS REFERENCE TO RELATED APPLICATIONS

The present disclosure is a U.S. National Stage entry under 35 U.S.C. §371 based on International Application No. PCT/EP2012/072246, filed Nov. 9, 2012 which was published under PCT Article 21(2) and which claims priority to German Patent Application No. DE 10 2011 089 578.7 filed on Dec. 22, 2011, which is incorporated herein by reference in its entirety.

### TECHNICAL FIELD

The agents contemplated herein relate to the technical field of the temporary reshaping of keratin-containing fibers, in particular human hair. It relates in particular to agents for hair treatment containing a special polymer combination, to the use of these agents for the temporary shaping and/or for the care of keratin-containing fibers and to aerosol sprays or aerosol foams based on these agents.

### BACKGROUND

The most important property of an agent for temporarily shaping keratinic fibers, also referred to below as a styling agent, consists in the first instance in giving the treated fibers as strong a hold as possible in the created shape. In addition to having a high degree of hold, styling agents also have to satisfy a whole series of further requirements. These can be broken down in approximate terms into properties of the hair, properties of the individual formulation, e.g. properties of the foam, gel or sprayed aerosol, and properties relating to the handling of the styling agent, particular importance being attached to the properties of the hair. These include in particular moisture resistance, low stickiness and a balanced conditioning effect. As far as possible a styling agent should furthermore be universally suitable for all hair types.

In order to meet the diverse requirements, a large number of synthetic polymers for use in styling agents have already been developed. The polymers can be divided into cationic, anionic, non-ionic and amphoteric film-forming and/or strengthening polymers. Even when used in small amounts, the polymers ideally form a polymer film when applied to the hair, which on the one hand gives the hairstyle a strong hold but on the other is sufficiently flexible not to break under stress. If the polymer film is too brittle, film flakes or residues are formed which detach when the hair is moved and give the impression that the user of the corresponding styling agent has dandruff. Moreover, hair coated with a strengthening polymer often feels coarse.

Furthermore, in addition to having a strong hold, the temporarily shaped hair should also look healthy and natural. The shine of the hair has an important part to play here. For that reason shine enhancers are often added in an adequate amount to the hair styling agents. These shine enhancers are oils or shine-imparting pigments such as mica particles, for example. Shine-imparting pigments have the disadvantage that over time they detach from the hair and after a while appear on clothing or facial skin, for example. Oils make the hair heavier and in some cases lead to an inferior adhesion of the film-forming or strengthening polymers to the hair. This can result in the disadvantage that the created hairstyle cannot be fixed for a sufficient length of time by the film-forming or strengthening polymers. The hairstyle drops out more quickly.

### SUMMARY

An agent for treating keratin-containing fibers is provided, which comprises a cosmetically acceptable carrier, and

(a) at least one strengthening anionic copolymer, comprising at least one structural unit of formula (I) and at least one structural unit of formula (II) and at least one structural unit of formula (III)

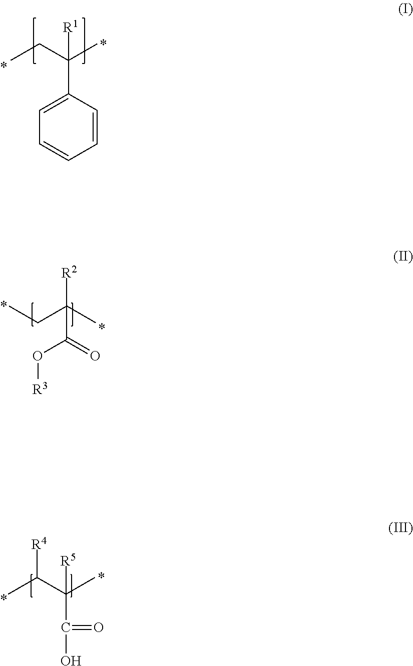

in which, R¹ denotes a hydrogen atom or a methyl group, R² denotes a hydrogen atom or a methyl group, R³ denotes a branched (C₁ to C₂₀) alkyl group, a branched (C₂ to C₆) hydroxyalkyl group or a *—(CH₂CH₂O)ₙ—R⁶ group where R⁶=branched (C₁ to C₂₀) alkyl and n=1 to 30, and R⁴ and R⁵ each denote a hydrogen atom, or one of the two residues denotes a hydrogen atom and the other denotes a methyl group, and

(b) at least one polymer comprising at least one structural unit of formula (M1)

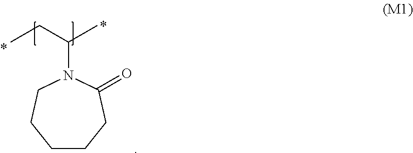

Also provided is a method for treating keratin-containing fibers in which a dispensing device is used to apply the agent to the keratin-containing fibers as a spray.

### DETAILED DESCRIPTION

One object was to provide an agent for the temporary shaping of keratin-containing fibers, which has a high degree of hold, does not dull the keratin-containing fibers and brings about good sensory qualities in the keratin-containing fibers (in particular a soft hair feel).

Surprisingly it has now been found that this can be achieved through a special combination of polymers.

In one embodiment, an agent for treating keratin-containing fibers is provided which comprises: a cosmetically acceptable carrier, and
(a) at least one strengthening anionic copolymer, comprising at least one structural unit of formula (I) and at least one structural unit of formula (II) and at least one structural unit of formula (III)

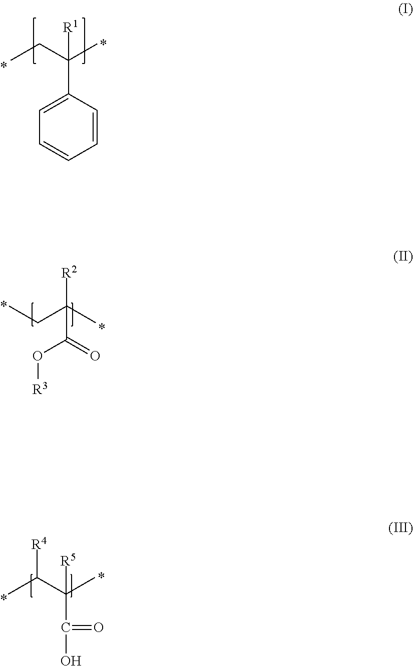

in which
R¹ denotes a hydrogen atom or a methyl group,
R² denotes a hydrogen atom or a methyl group,
R³ denotes a branched (C₁ to C₂₀) alkyl group, a branched (C₂ to C₆) hydroxyalkyl group or a *—(CH₂CH₂O)ₙ—R⁶ group where R⁶=branched (C₁ to C₂₀) alkyl and n=1 to 30,
R⁴ and R⁵ each denote a hydrogen atom, or one of the two residues denotes a hydrogen atom and the other denotes a methyl group, and
(b) at least one polymer comprising at least one structural unit of formula (M1)

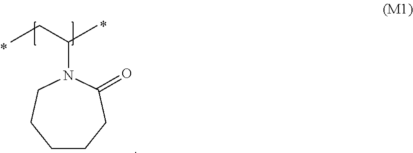

In the above formulae and in all subsequent formulae, a chemical bond marked with the symbol * denotes a “free valence” of the corresponding structural fragment. Further structural fragments bind to this “free valence” in the corresponding copolymer. These are structural fragments of the polymer in question that have already been described and optionally additionally further structural fragments.

Strengthening polymers contribute to the hold and/or to establishing volume and fullness in the hairstyle as a whole. These polymers are simultaneously also film-forming polymers and are therefore generally typical substances for hair treatment agents used for shaping, such as hair fixing agents, hair foams, hair waxes, hair sprays. Film formation may be entirely localized here and may bond only a few fibers together. The curl retention test is frequently used as a test method for the strengthening effect of a polymer.

Film-forming polymers are understood to be polymers which leave behind a continuous film on the skin, hair or nails when they dry. Such film formers can be used in a wide range of cosmetic products, such as for example face masks, make-up, hair fixing agents, hair sprays, hair gels, hair waxes, hair masks, shampoos or nail varnishes. Such polymers are preferred in particular which have an adequate solubility in water or water/alcohol mixtures in order for them to be present in the agent in completely dissolved form. The film-forming polymers can be of synthetic or natural origin.

Film-forming polymers are also understood to be polymers which when used in an about 0.01 to about 20 wt. % aqueous, alcoholic or aqueous-alcoholic solution are capable of depositing a transparent polymer film on the hair.

Within the meaning of the invention “crosslinked” or “crosslinking” is understood to mean the linking together of polymer chains by covalent chemical bonding to form a network. This covalent linking of the polymer chains is carried out by a bridging polysiloxane molecule fragment. The crosslinking polysiloxane molecule fragment binds in each case by means of covalent chemical bonding to the polymer chains that are bridged by the molecule fragment.

An anionic polymer means a polymer which in a protic solvent under standard conditions bears structural units having anionic groups needing to be offset by counterions to maintain electrical neutrality and no structural units having permanently cationic or cationizable groups. Anionic groups include carboxyl and sulfonic acid groups.

The strengthening anionic copolymer comprises at least one structural unit of formula (I) and at least one structural unit of formula (II) and at least one structural unit of formula (III)

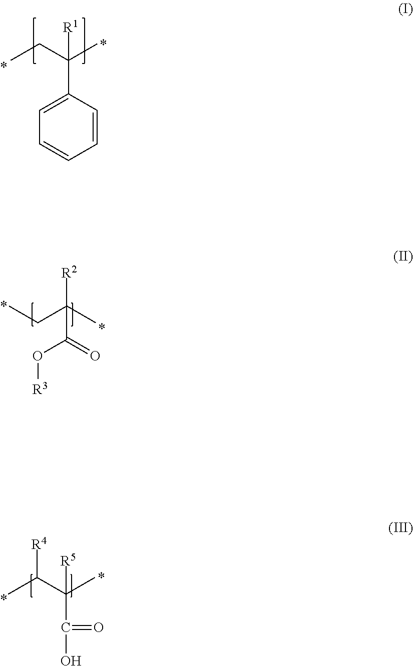

as defined above. In preferred agents the residue R¹ denotes a hydrogen atom, making styrene a preferred monomer in the copolymers.

Preferred agents contain as structural unit (II) esters of acrylic acid or methacrylic acid with branched alcohols, acrylic acid esters being preferred, such that in preferred agents R² denotes a hydrogen atom.

R³ denotes a branched (C₁ to C₂₀) alkyl group, a branched (C₂ to C₆) hydroxyalkyl group or a *—(CH₂CH₂O)ₙ—R⁶ group where R⁶=branched (C₁ to C₂₀) alkyl and n=1 to 30, wherein isopropyl, sec-butyl, tert-butyl and isooctyl(ethylhexyl) groups are preferred as branched alkyl groups.

It has proved advantageous for the hold and shine of hair treated with the agents if the strengthening anionic copolymers have not only a structural unit of formula (II) in which acrylic acid or methacrylic acid is esterified with branched alcohols but also a structural unit in which acrylic acid or methacrylic acid are esterified with unbranched alcohols.

Agents are therefore characterized in that the strengthening anionic copolymer comprises at least one structural unit of formula (I) and at least one structural unit of formula (IIa) and at least one structural unit of formula (IIb) and at least one structural unit of formula (III)

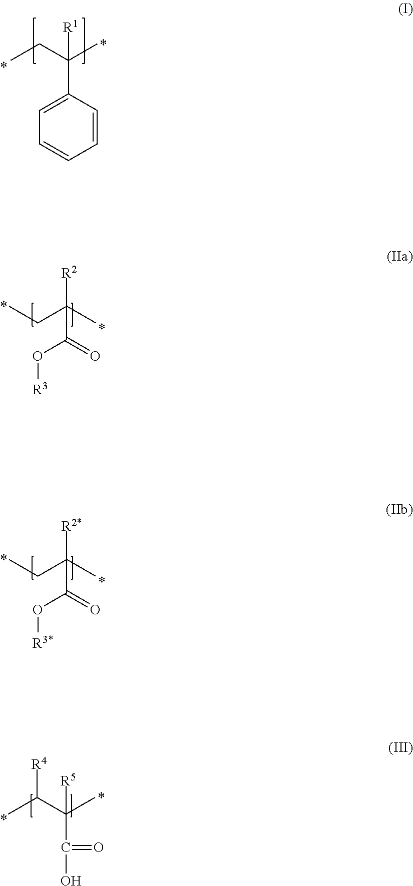

in which
R¹ denotes a hydrogen atom,
R² denotes a hydrogen atom,
R³ denotes an ethylhexyl group,
R²* denotes a hydrogen atom,
R³* denotes a butyl group,
R⁴ and R⁵ each denote a hydrogen atom, or one of the two residues denotes a hydrogen atom and the other denotes a methyl group.

As the third required structural unit the copolymers used in the agents contain acrylic acid (R⁴ and R⁵=—H) or methacrylic acid (R⁴=—CH₃, R⁵=—H).

Most particularly preferred agents contain as strengthening anionic polymer(s):
copolymers of styrene with isopropyl acrylate and acrylic acid
copolymers of styrene with sec-butyl acrylate and acrylic acid
copolymers of styrene with tert-butyl acrylate and acrylic acid
copolymers of styrene with ethylhexyl acrylate and acrylic acid
copolymers of styrene with isopropyl acrylate and methyl acrylate and acrylic acid
copolymers of styrene with sec-butyl acrylate and methyl acrylate and acrylic acid
copolymers of styrene with tert-butyl acrylate and methyl acrylate and acrylic acid
copolymers of styrene with ethylhexyl acrylate and methyl acrylate and acrylic acid
copolymers of styrene with isopropyl acrylate and ethyl acrylate and acrylic acid
copolymers of styrene with sec-butyl acrylate and ethyl acrylate and acrylic acid
copolymers of styrene with tert-butyl acrylate and ethyl acrylate and acrylic acid
copolymers of styrene with ethylhexyl acrylate and ethyl acrylate and acrylic acid
copolymers of styrene with isopropyl acrylate and propyl acrylate and acrylic acid
copolymers of styrene with sec-butyl acrylate and propyl acrylate and acrylic acid
copolymers of styrene with tert-butyl acrylate and propyl acrylate and acrylic acid
copolymers of styrene with ethylhexyl acrylate and propyl acrylate and acrylic acid
copolymers of styrene with isopropyl acrylate and isopropyl acrylate and acrylic acid
copolymers of styrene with sec-butyl acrylate and isopropyl acrylate and acrylic acid
copolymers of styrene with tert-butyl acrylate and isopropyl acrylate and acrylic acid
copolymers of styrene with ethylhexyl acrylate and isopropyl acrylate and acrylic acid
copolymers of styrene with isopropyl acrylate and n-butyl acrylate and acrylic acid
copolymers of styrene with sec-butyl acrylate and n-butyl acrylate and acrylic acid
copolymers of styrene with tert-butyl acrylate and n-butyl acrylate and acrylic acid
copolymers of styrene with ethylhexyl acrylate and n-butyl acrylate and acrylic acid
copolymers of styrene with isopropyl acrylate and methacrylic acid
copolymers of styrene with sec-butyl acrylate and methacrylic acid
copolymers of styrene with tert-butyl acrylate and methacrylic acid
copolymers of styrene with ethylhexyl acrylate and methacrylic acid
copolymers of styrene with isopropyl acrylate and methyl acrylate and methacrylic acid
copolymers of styrene with sec-butyl acrylate and methyl acrylate and methacrylic acid
copolymers of styrene with tert-butyl acrylate and methyl acrylate and methacrylic acid
copolymers of styrene with ethylhexyl acrylate and methyl acrylate and methacrylic acid
copolymers of styrene with isopropyl acrylate and ethyl acrylate and methacrylic acid
copolymers of styrene with sec-butyl acrylate and ethyl acrylate and methacrylic acid
copolymers of styrene with tert-butyl acrylate and ethyl acrylate and methacrylic acid
copolymers of styrene with ethylhexyl acrylate and ethyl acrylate and methacrylic acid
copolymers of styrene with isopropyl acrylate and propyl acrylate and methacrylic acid
copolymers of styrene with sec-butyl acrylate and propyl acrylate and methacrylic acid
copolymers of styrene with tert-butyl acrylate and propyl acrylate and methacrylic acid
copolymers of styrene with ethylhexyl acrylate and propyl acrylate and methacrylic acid
copolymers of styrene with isopropyl acrylate and isopropyl acrylate and methacrylic acid
copolymers of styrene with sec-butyl acrylate and isopropyl acrylate and methacrylic acid
copolymers of styrene with tert-butyl acrylate and isopropyl acrylate and methacrylic acid
copolymers of styrene with ethylhexyl acrylate and isopropyl acrylate and methacrylic acid
copolymers of styrene with isopropyl acrylate and n-butyl acrylate and methacrylic acid
copolymers of styrene with sec-butyl acrylate and n-butyl acrylate and methacrylic acid
copolymers of styrene with tert-butyl acrylate and n-butyl acrylate and methacrylic acid
copolymers of styrene with ethylhexyl acrylate and n-butyl acrylate and methacrylic acid

The agent contains the first strengthening anionic copolymer preferably in an amount from about 0.01 to about 29.99 wt. %, particularly preferably from about 0.1 to about 14.9 wt. %, most particularly preferably from about 0.1 to about 9.5 wt. % and most preferably from about 0.2 to about 7.0 wt. %, relative in each case to the total weight of the agent.

The agent mandatorily contains as component (b) a polymer having at least one caprolactam structural unit of formula (M1) above.

The polymers of component (b) are preferably contains in the agent in an amount from about 0.05 to about 15.0 wt. %, particularly preferably about out 0.1 to 7.0 wt. %, relative in each case to the total weight of the agent.

The polymers of component (b) of the agent are preferably non-ionic or cationic. Cationic polymers are understood to be polymers having a group in the main and/or side chain which can be “temporarily” or “permanently” cationic. Polymers which have a cationic group irrespective of the pH of the agent are described as “permanently cationic”. These are generally polymers containing a quaternary nitrogen atom, in the form of an ammonium group for example. Preferred cationic groups are quaternary ammonium groups. In particular, polymers in which the quaternary ammonium group is bound via a C₁₋₄ hydrocarbon group to a polymer main chain synthesized from acrylic acid, methacrylic acid or derivatives thereof have proved to be suitable.

Further cationic, strengthening polymers which can preferably be used in the agents are the “temporarily cationic” polymers. These polymers are preferably cationic polymers. They conventionally contain an amino group which is present in protic solvents as a protonated ammonium group and hence is cationic. The degree of cationization is dependent on the pH.

The polymers of component (b) of the agent are particularly preferably non-ionic or temporarily cationic.

A homopolymer of N-vinylcaprolactam is preferably suitable. Such polymers are non-ionic and are sold for example by BASF SE under the trade name Luviskol Plus (INCI name: Polyvinylcaprolactam; 50 wt. % active substance in ethanol).

Particularly preferred agents are characterized in that the polymer of component (b) comprises at least one structural unit of formula (M1) and at least one structural unit of formula (M2) and at least one structural unit of formula (M3),

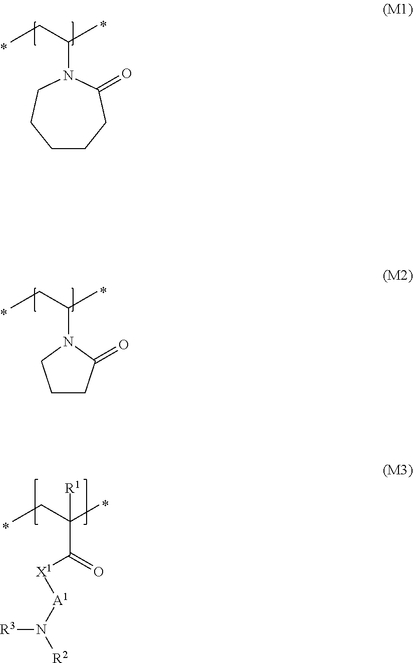

R¹ denotes a hydrogen atom or a methyl group,
X¹ denotes an oxygen atom or an NH group,
A¹ denotes an ethane-1,2-diyl, propane-1,3-diyl or butane-1,4-diyl group,
R² and R³ independently of one another denote a (C₁ to C₄) alkyl group.

R¹ in formula (M3) preferably denotes a methyl group.

Examples of (C₁ to C₄) alkyl groups according to formula (M3) are methyl, ethyl, isopropyl, n-propyl, n-butyl, sec-butyl, isobutyl and tert-butyl. R² and R³ in formula (M3) preferably denote a methyl group.

Most particularly suitable polymers of this embodiment have at least one structural unit of formula (M3) in which A¹ denotes an ethane-1,2-diyl group and X¹ denotes an oxygen atom. Such a polymer preferably in turn comprises preferably at least one structural unit of formula (M1) and at least one structural unit of formula (M2) and at least one structural unit of formula (M3-1)

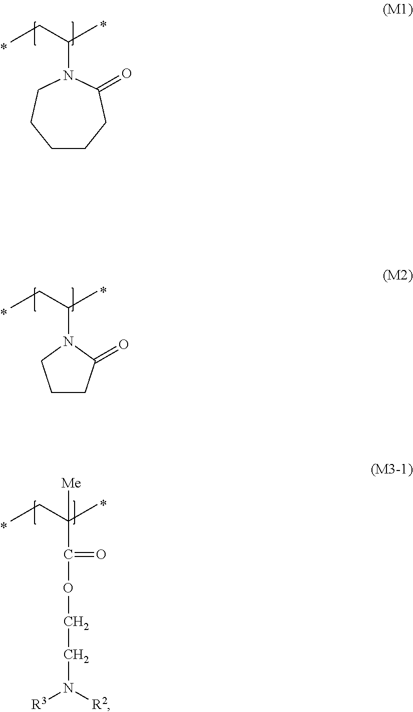

in which R² and R³ independently of one another denote a (C₁ to C₄) alkyl group, in particular methyl. Examples of (C₁ to C₄) alkyl groups according to formula (M3-1) are methyl, ethyl, isopropyl, n-propyl, n-butyl, sec-butyl, isobutyl and tert-butyl.

The agent most particularly preferably contains as component (b) of the agent a terpolymer of N-vinylcaprolactam, N-vinylpyrrolidone and N,N-dimethylaminoethyl methacrylate. Such copolymers are obtainable from Ashland for example under the trade name Advantage LC-E (INCI name: Vinylcaprolactam/VP/Dimethylaminoethylmethacrylate Copolymer, Laurylpyrrolidone; 37 wt. % active substance in ethanol with addition of N-laurylpyrrolidone), Advantage S (INCI name: Vinylcaprolactam/VP/Dimethylaminoethylmethacrylate Copolymer, 100 wt. % active substance, powder) or Advantage LC-A (INCI name: Vinylcaprolactam/VP/Dimethylaminoethylmethacrylate Copolymer; 37 wt. % active substance in ethanol).

In the context of a likewise most preferred embodiment the agents contain as component (b) at least one polymer comprising at least one structural unit of formula (M2) and at least one structural unit of formula (M3),

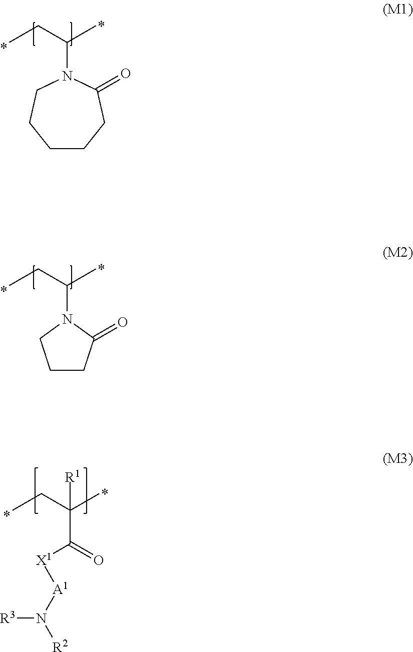

R¹ denotes a hydrogen atom or a methyl group,
X¹ denotes an NH group,
A¹ denotes a propane-1,3-diyl group,
R² and R³ independently of one another denote a (C₁ to C₄) alkyl group, in particular methyl.

Such polymers of component (b) comprising at least one structural unit of formula (M1) and at least one structural unit of formula (M2) and at least one structural unit of formula (M3-2) are preferred in turn

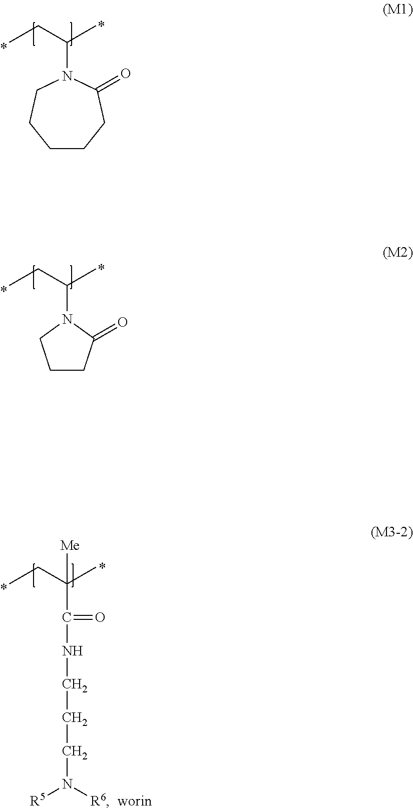

in which
R⁵ and R⁶ independently of one another denote a (C₁ to C₄) alkyl group, in particular methyl. The agent of this embodiment most particularly preferably contains as the polymer of component (b) of the agent a terpolymer of N-vinylpyrrolidone, N-vinylcaprolactam and N,N-dimethylaminopropyl methacrylamide. Such copolymers are available for example from ISP under the trade name Aquaflex SF 40 (INCI name: VP/Vinyl Caprolactam/DMAPA Acrylates Copolymer, Alcohol Denat.; 38-42 wt. % active substance in ethanol).

In the context of a further embodiment the agent contains as the polymer of component (b) a cationic polymer comprising at least one structural unit of formula (M1) and at least one structural unit of formula (M2) and at least one structural unit of formula (M3) and at least one structural unit of formula (M4),

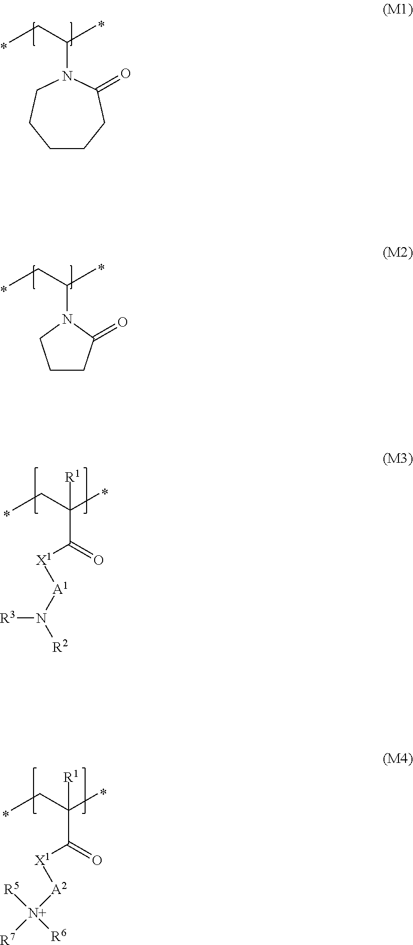

in which
R¹ and R⁴ independently of one another denote a hydrogen atom or a methyl group,
X¹ and X² independently of one another denote an oxygen atom or an NH group,
A¹ and A² independently of one another denote an ethane-1,2-diyl, propane-1,3-diyl or butane-1,4-diyl group,
R², R³, R⁵ and R⁶ independently of one another denote a (C₁ to C₄) alkyl group,
R⁷ denotes a (C₈ to C₃₀) alkyl group

A cationic polymer that is preferably included in the agent as component (b) in the context of this embodiment comprises at least one structural unit of formula (M1), at least one structural unit of formula (M2), at least one structural unit of formula (M3-2) and at least one structural unit of formula (M4-1),

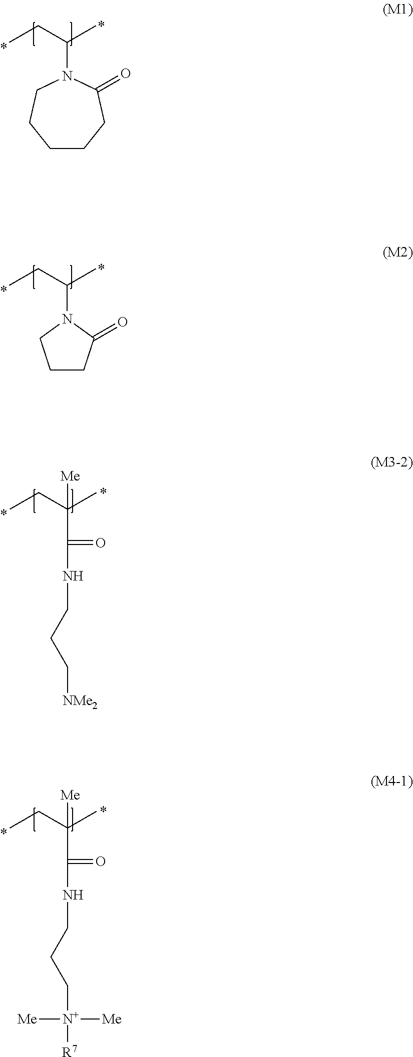

in which R⁷ denotes octyl(capryl), decyl(caprinyl), dodecyl(lauryl), tetradecyl(myristyl), hexadecyl(cetyl), octadecyl(stearyl), eicosyl(arachyl) or docosyl(behenyl).

A preferred cationic polymer is in turn the copolymer of N-vinylpyrrolidone, N-vinylcaprolactam, N-(3-dimethylaminopropyl)methacrylamide and 3-(methacryloylamino)propyl lauryl dimethylammonium chloride (INCI name: Polyquaternium-69), which is sold for example by ISP under the trade name AquaStyle® 300 (28-32 wt. % active substance in an ethanol-water blend, molecular weight 350,000).

To achieve the object it has in turn proved preferable additionally to add to the agent (c) at least one copolymer comprising at least one structural unit of formula (M5)

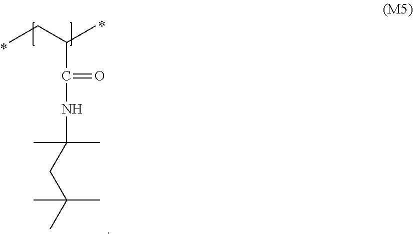

The copolymers of component (c) contained in the agent in this embodiment of the invention are preferably included in an amount from about 0.1 to about 10.0 wt. %, particularly preferably about 0.3 to about 8.0 wt. %, relative in each case to the total weight of the agent.

In the context of a preferred embodiment of the invention the agent additionally contains as component (c) at least one copolymer having at least one structural unit of formula (M5), at least one structural unit of formula (M6) and at least one structural unit of formula (M7),

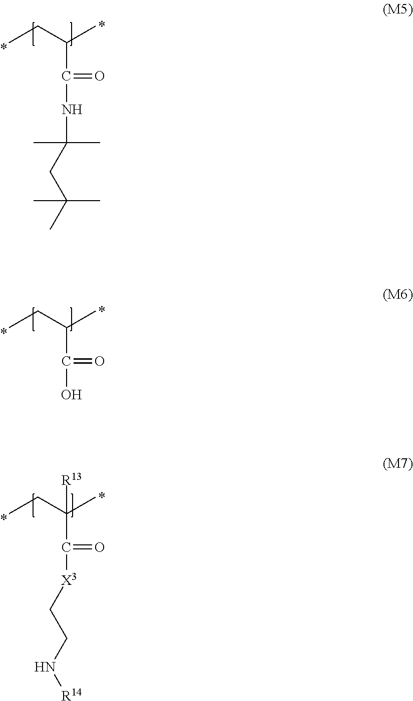

in which X³ denotes an oxygen atom or an NH group,
R¹³ denotes a hydrogen atom or a methyl group and
R¹⁴ denotes an alkyl group having 4 carbon atoms (in particular n-butyl, sec-butyl, isobutyl or tert-butyl). It is particularly preferable in turn for the copolymer of component (c) additionally to contain, in addition to the above structural units of formulae (M5), (M6) and (M7), at least one structural unit of formula (M8)

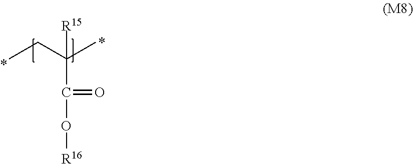

in which
R¹⁵ denotes a hydrogen atom or a methyl group
R¹⁶ denotes a (C₁ to C₄) alkyl group (in particular a methyl group or an ethyl group).

Preferred copolymers of component (c) of this type are selected from the group formed from:
copolymers of i) acrylic acid, ii) at least one monomer of (C₁ to C₄) alkyl acrylates and/or (C₁ to C₄) methalkyl acrylates, iii) C₄ alkyl aminoethyl methacrylate and iv) C₈ alkyl acrylamide.

An example of a copolymer of component (c) that can particularly preferably be used in the context of this embodiment is the polymer with the INCI name Octylacrylamide/Acrylates/Butylaminoethylmethacrylate Copolymer, which is obtainable in powder form from Akzo Nobel under the trade name AMPHOMER® 28-4910.

The effects can be increased by the addition of alkyl esters of hydroxy acids. Agents that are preferred are characterized in that they contain, relative in each case to the total weight of the agent, about 0.05 to about 5 wt. %, preferably about 0.1 to about 2.5 wt. % and in particular about 0.15 to about 0.5 wt. % of at least one alkyl ester of a hydroxy acid.

Most particularly preferred alkyl esters of hydroxy acids are the (C₂ to C₆) trialkyl citrates.

It is therefore preferable for the agents additionally to contain at least one compound of formula (E),

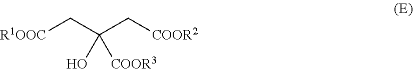

in which
R¹, R² and R³ independently of one another denote a (C₂ to C₆) alkyl group.

Examples of a (C₂ to C₆) alkyl group according to formula (E) are methyl, ethyl, isopropyl, n-propyl, n-butyl, sec-butyl, isobutyl, tert-butyl, n-pentyl, neopentyl, isopentyl, n-hexyl.

Triethyl citrate has proved to be a particularly preferred compound of formula (E).

The agent contains the compounds of formula (E) preferably in an amount from about 0.01 to about 1 wt. %, in particular from about 0.05 to about 0.3 wt. %, relative in each case to the total weight of the agent. Agents that are preferred in particular are characterized in that they contain, relative in each case to the total weight of the agent, about 0.05 to about 5 wt. %, preferably about 0.1 to about 2.5 wt. % and in particular about 0.15 to about 0.5 wt. % of triethyl citrate.

The agents contain the ingredients and active agents in a cosmetically acceptable carrier.

Preferred cosmetically acceptable carriers are aqueous, alcoholic or aqueous-alcoholic media having preferably at least about 10 wt. % water, relative to the total weight of the agent. The low alcohols having 1 to 4 carbon atoms that are conventionally used for cosmetic purposes, such as for example ethanol and isopropanol, can be included in particular as alcohols.

In the context of a preferred embodiment, the agent therefore additionally contains at least one alcohol having 2 to 6 carbon atoms and 1 to 3 hydroxyl groups. This additional alcohol is in turn preferably selected from at least one compound of the group formed from ethanol, ethylene glycol, isopropanol, 1,2-propylene glycol, 1,3-propylene glycol, glycerol, n-butanol, 1,3-butylene glycol. A most particularly preferred alcohol is ethanol.

Most particularly preferred agents are characterized in that they contain, relative in each case to the total weight of the agent, about 10 to about 70 wt. %, preferably about 20 to about 65 wt. %, more preferably about 30 to about 60 wt. % and in particular about 35 to about 50 wt. % of ethanol and/or isopropanol.

The additional alcohol having 2 to 6 carbon atoms and 1 to 3 hydroxyl groups is preferably contained in the agent (in particular in the presence of at least one propellant) in an amount from about 40 wt. % to about 65 wt. %, in particular from about 40 wt. % to about 50 wt. %, relative in each case to the total weight of the agent.

Organic solvents or a mixture of solvents with a boiling point below 400° C. can be included as additional co-solvents in an amount from about 0.1 to about 15 percent by weight, preferably from about 1 to about 10 percent by weight, relative to the total agent. Unbranched or branched hydrocarbons, such as pentane, hexane, isopentane, and cyclic hydrocarbons, such as cyclopentane and cyclohexane, are particularly suitable as additional co-solvents. Further, particularly preferred water-soluble solvents are polyethylene glycol and propylene glycol in an amount of up to about 30 wt. % relative to the total agent.

In particular, the addition of propylene glycol and/or polyethylene glycol and/or polypropylene glycol increases the flexibility of the polymer film formed on application of the agent. If a flexible hold is desired, the agents therefore preferably contain about 0.01 to about 30 wt. % of polyethylene glycol and/or polypropylene glycol, relative to the total weight of the agent.

The agents preferably have a pH from about 2 to about 11. The pH range between about 2 and about 8 is particularly preferred. Unless otherwise specified, within the meaning of this document the pH values stated relate to the pH at 25° C.

By preference the agents additionally contain at least one surfactant, wherein non-ionic, anionic, cationic and ampholytic surfactants are suitable in particular. The group of ampholytic or amphoteric surfactants comprises zwitterionic surfactants and ampholytes. the surfactants can already have an emulsifying action. The use of at least one non-ionic surfactant and/or at least one cationic surfactant is preferred within the context of this embodiment of the invention.

The additional surfactants are preferably contained in the agent in an amount from about 0.01 wt. % to about 5 wt. %, particularly preferably from about 0.05 wt. % to about 0.5 wt. %, relative in each case to the total weight of the agent.

It has proved particularly preferable for the agents additionally to contain at least one non-ionic surfactant.

Non-ionic surfactants contain as a hydrophilic group a polyol group, a polyalkylene glycol ether group or a combination of a polyol and polyglycol ether group, for example. Such compounds are for example
addition products of about 2 to about 100 mol of ethylene oxide and/or about 1 to about 5 mol of propylene oxide with linear and branched fatty alcohols having 8 to 30 C atoms, with fatty acids having 8 to 30 C atoms and with alkylphenols having 8 to 15 C atoms in the alkyl group,
addition products of about 2 to about 50 mol of ethylene oxide and/or about 1 to about 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as for example the types available under the commercial names Dehydrol® LS, Dehydrol® LT (Cognis),
C₁₂-C₃₀ fatty acid monoesters and diesters of addition products of about 1 to about 30 mol of ethylene oxide with glycerol,
addition products of about 5 to about 60 mol of ethylene oxide with castor oil and hydrogenated castor oil,
polyol fatty acid esters, such as for example the commercial product HYDAGEN® HSP (Cognis) or SOVERMOL types (Cognis),
alkoxylated triglycerides,
alkoxylated fatty acid alkyl esters of formula (T-I)

R¹CO—(OCH₂CHR²)_{w}OR³ (T-I)
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms and w denotes numbers from 1 to 20,
amine oxides,
hydroxy mixed ethers, such as are described for example in DE-OS 19738866,
sorbitan fatty acid esters and addition products of ethylene oxide with sorbitan fatty acid esters such as for example polysorbates,
sugar fatty acid esters and addition products of ethylene oxide with sugar fatty acid esters,
addition products of ethylene oxide with fatty acid alkanol amides and fatty amines,
sugar surfactants of the alkyl and alkenyl oligoglycoside type according to formula (T-II),

R⁴O—[G]ₚ (T-II)
in which R⁴ denotes an alkyl or alkenyl residue having 4 to 22 carbon atoms, G denotes a sugar residue having 5 or 6 carbon atoms and p denotes numbers from about 1 to about 10. They can be obtained by means of the relevant methods of preparative organic chemistry. The preferred alkyl and/or alkenyl oligoglycosides are alkyl and/or alkenyl oligoglucosides. The index value p in the general formula (T-II) indicates the degree of oligomerization (DP), i.e. the distribution of mono- and oligoglycosides, and denotes a number between about 1 and about 10. While p in the individual molecule must always be an integer and above all can assume the values p=about 1 to about 6 here, the value p for a particular alkyl oligoglycoside is a calculated quantity determined by analysis, which in most cases is a fraction. Alkyl and/or alkenyl oligoglycosides having an average degree of oligomerization p from about 1.1 to about 3.0 are preferably used. From an application-oriented perspective, alkyl and/or alkenyl oligoglycosides having a degree of oligomerization below about 1.7 and in particular between about 1.2 and about 1.4 are preferred. Alkyl oligoglucosides based on hydrogenated C_{12/14} coconut alcohol with a DP from about 1 to about 3 are preferred.

The alkylene oxide addition products with saturated linear fatty alcohols and fatty acids each containing about 2 to about 100 mol of ethylene oxide per mol of fatty alcohol or fatty acid have proved to be most particularly preferred non-ionic surfactants. Preparations having outstanding properties are likewise obtained if they contain C₁₂-C₃₀ fatty acid mono- and diesters of addition products of about 1 to about 30 mol of ethylene oxide with glycerol and/or addition products of about 5 to about 60 mol of ethylene oxide with castor oil and hydrogenated castor oil as non-ionic surfactants.

Both products having a “normal” homolog distribution and those having a narrow homolog distribution can be used for the surfactants that are addition products of ethylene and/or propylene oxide with fatty alcohols or derivatives of these addition products. “Normal” homolog distribution refers to mixtures of homologs which are obtained by reacting fatty alcohol and alkylene oxide using alkali metals, alkali metal hydroxides or alkali metal alcoholates as catalysts. Narrow homolog distributions on the other hand are obtained when for example hydrotalcites, alkaline-earth metal salts of ether carboxylic acids, alkaline-earth metal oxides, hydroxides or alcoholates are used as catalysts. The use of products having a narrow homolog distribution can be preferred.

The agents most particularly preferably contain as the surfactant at least one addition product of about 15 to about 100 mol of ethylene oxide, in particular of about 15 to about 50 mol of ethylene oxide, with a linear or branched (in particular linear) fatty alcohol having 8 to 22 carbon atoms. This is particularly preferably Ceteareth-15, Ceteareth-25 or Ceteareth-50, which are sold respectively as EUMULGIN® CS 15 (COGNIS), Cremophor A25 (BASF SE) and EUMULGIN® CS 50 (COGNIS).

All anionic surface-active substances which are suitable for use on the human body are suitable in principle as anionic surfactants. These are characterized by a water-solubilizing anionic group such as for example a carboxylate, sulfate, sulfonate or phosphate group and a lipophilic alkyl group having approximately 8 to 30 C atoms. The molecule can additionally contain glycol or polyglycol ether groups, ester, ether and amide groups and hydroxyl groups. Examples of suitable anionic surfactants, each in the form of the sodium, potassium and ammonium salts as well as the mono-, di- and trialkanolammonium salts having 2 to 4 C atoms in the alkanol group, are
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
mixtures of surface-active hydroxyl sulfonates,
sulfated hydroxyalkyl polyethylene and/or hydroxyalkylene propylene glycol ethers,
sulfonates of unsaturated fatty acids having 8 to 24 C atoms and 1 to 6 double bonds,
esters of tartaric acid and citric acid with alcohols that are addition products of approximately 2 to 15 molecules of ethylene oxide and/or propylene oxide with fatty alcohols having 8 to 22 C atoms,
alkyl and/or alkenyl ether phosphates of the formula (T-V),

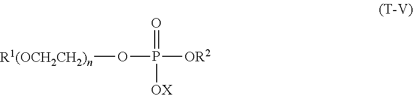

in which R¹ preferably denotes an aliphatic hydrocarbon residue having 8 to 30 carbon atoms, R² denotes hydrogen, a (CH₂CH₂O)ₙR¹ residue or X, n denotes numbers from 1 to 10 and X denotes hydrogen, an alkali or alkaline-earth metal or NR³R⁴R⁵R⁶, with R³ to R⁶ independently of one another denoting hydrogen or a C₁ to C₄ hydrocarbon residue,
sulfated fatty acid alkylene glycol esters of formula (T-VI)

R⁷CO(AlkO)ₙSO₃M (T-VI)
in which R⁷CO denotes a linear or branched, aliphatic, saturated and/or unsaturated acyl residue having 6 to 22 C atoms, Alk denotes CH₂CH₂, CHCH₃CH₂ and/or CH₂CHCH₃, n denotes numbers from 0.5 to 5 and M denotes a cation,
amide ether carboxylic acids,
condensation products of C₈ to C₃₀ fatty alcohols with protein hydrolysates and/or amino acids and derivatives thereof, which are known to the person skilled in the art as protein fatty acid condensates, such as for example the LAMPEON® types, GLUADIN® types, HOSTAPON® KCG or AMISOFT® types.

Also suitable for use are cationic surfactants of the quaternary ammonium compound, esterquat and amidoamine type. Preferred quaternary ammonium compounds are ammonium halides, in particular chlorides and bromides, such as alkyl trimethylammonium chlorides, dialkyl dimethylammonium chlorides and trialkyl methylammonium chlorides. The long alkyl chains of these surfactants preferably have 10 to 18 carbon atoms, such as for example in cetyl trimethylammonium chloride, stearyl trimethylammonium chloride, distearyl dimethylammonium chloride, lauryl dimethylammonium chloride, lauryl dimethyl benzylammonium chloride and tricetyl methylammonium chloride. Further preferred cationic surfactants are the imidazolium compounds known under the INCI names Quaternium-27 and Quaternium-83.

Surface-active compounds classed as zwitterionic surfactants are those bearing at least one quaternary ammonium group and at least one —COO⁽⁻⁾ or —SO₃⁽⁻⁾ group in the molecule. Particularly suitable zwitterionic surfactants are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytes are understood to be surface-active compounds which in addition to a C₈-C₂₄ alkyl or acyl group contain at least one free amino group and at least one —COOH or —SO₃H group in the molecule and are capable of forming internal salts. Examples of suitable ampholytes are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytes are N-cocoalkyl aminopropionate, cocoacylaminoethyl aminopropionate and C₁₂-C₁₈ acyl sarcosine.

The agents can furthermore contain the auxiliary substances and additives that are conventionally added to customary styling agents.

Suitable auxiliary substances and additives include in particular additional care substances.

The agent can for example contain at least one protein hydrolysate and/or a derivative thereof as a care substance.

Protein hydrolysates are mixtures of products which are obtained by acidically, basically or enzymatically catalyzed breakdown of proteins. As used herein, the term protein hydrolysates is also understood to include total hydrolysates and individual amino acids and derivatives thereof as well as mixtures of different amino acids. As used herein. the term protein hydrolysates is furthermore understood to include polymers synthesized from amino acids and amino acid derivatives. β-Amino acids and derivatives thereof can of course also be used. The molecular weight of the protein hydrolysates for use is between about 75 daltons, the molecular weight for glycine, and about 200,000 daltons; the molecular weight is preferably about 75 to about 50,000 daltons and particularly preferably about 75 to about 20,000 daltons.

Protein hydrolysates of both plant and animal or marine or synthetic origin can be used.

Although the use of protein hydrolysates as such is preferred, amino acid mixtures obtained by other means can optionally also be used in their place.

The protein hydrolysates can be contained in the agents in concentrations for example from about 0.01 wt. % to about 20 wt. %, preferably from about 0.05 wt. % to about 15 wt. % and most particularly preferably in amounts from about 0.05 wt. % to about 5 wt. %, relative in each case to the total weight of the agent.

The agent can furthermore contain at least one vitamin, provitamin, vitamin precursor and/or derivative thereof as a care substance.

Those vitamins, provitamins and vitamin precursors that are conventionally assigned to groups A, B, C, E, F and H are preferred.

The agents preferably contain vitamins, provitamins and vitamin precursors from groups A, B, C, E and H. Panthenol, pantolactone, pyridoxine and derivatives thereof as well as nicotinic acid amide and biotin are particularly preferred.

Like the addition of glycerol and/or propylene glycol, the addition of panthenol increases the flexibility of the polymer film formed on application of the agent. If a particularly flexible hold is desired, the agents can therefore contain panthenol instead of or in addition to glycerol and/or propylene glycol. In a preferred embodiment the agents contain panthenol, preferably in an amount from about 0.05 to about 10 wt. %, particularly preferably about 0.1 to about 5 wt. %, relative in each case to the total agent.

The agents can moreover contain at least one plant extract as a care substance. These extracts are conventionally produced by extraction of the entire plant. It can also be preferable in individual cases, however, to produce the extracts exclusively from flowers and/or leaves of the plant. The extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are most preferred.

The plant extracts can be used in both pure and diluted form. If they are used in diluted form they conventionally contain approximately about 2 to about 80 wt. % of active substance and as the solvent the extracting agent or mixture of extracting agents used to obtain them. It can furthermore be preferable to use mixtures of a plurality of different plant extracts, in particular two, in the agents. The agents preferably contain these care substances in amounts from about 0.001 to about 2, in particular from about 0.01 to about 0.5 wt. %, relative in each case to the total weight of the agent.

It has been found that through the addition of a UV filter both the agents themselves and also the treated fibers can be protected from damaging influences of UV radiation. Surprisingly the shine of the fibers treated with the agents is also improved and longer-lasting. At least one UV filter is therefore preferably added to the agent. The UV filters are conventionally included in amounts from about 0.01 to about 5 wt. %, relative to the total weight of the agent. Amounts from about 0.1 to about 2.5 wt. % are preferred. Agents that are preferred contain, relative in each case to the total weight of the agent, about 0.05 to about 5 wt. %, preferably about 0.1 to about 2.5 wt. % and in particular about 0.15 to about 0.5 wt. % of UV filter substances.

There are no general restrictions on the suitable UV filters in terms of their structure and their physical properties. In fact all UV filters that can be used in the cosmetics sector whose absorption maximum is in the UVA (315-400 nm), UVB (280-315 nm) or UVC (<280 nm) range are suitable. UV filters having an absorption maximum in the UVB range, in particular in the range from approximately 280 to approximately 300 nm, are particularly preferred.

The preferred UV filters can be selected for example from substituted benzophenones, p-aminobenzoic acid esters, diphenyl acrylic acid esters, cinnamic acid esters, salicylic acid esters, benzimidazoles, cinnamates and o-aminobenzoic acid esters.

UV filters whose molar extinction coefficient at the absorption maximum is above about 15,000, in particular above about 20,000, are preferred.

Most particularly preferred agents contain, relative in each case to the total weight of the agent, about 0.05 to about 5 wt. %, preferably about 0.1 to about 2.5 wt. % and in particular about 0.15 to about 0.5 wt. % of benzophenone-4 and/or isoamyl-p-methoxycinnamate.

In addition to the cited components, the agents can furthermore contain all active agents, additives and auxiliary substances known for such preparations.

Further active agents, auxiliary substances and additives are for example
thickening agents such as agar-agar, guar gum, alginates, xanthan gum, gum arabic, karaya gum, carob seed meal, linseed gums, dextrans, cellulose derivatives, for example methyl cellulose, hydroxyalkyl cellulose and carboxymethyl cellulose, starch fractions and derivatives such as amylose, amylopectin and dextrins, clays such as for example bentonite, fully synthetic hydrocolloids such as for example polyvinyl alcohol, and optionally crosslinked polyacrylates,
texturizing agents such as maleic acid and lactic acid,
perfume oils, dimethyl isosorbide and cyclodextrins,
defoaming agents such as silicones,
dyes to color the agent,
anti-dandruff active agents such as piroctone olamine, zinc omadine and climbazole,
substances to adjust the pH, such as for example conventional acids, in particular edible acids, and bases,
consistency modifiers such as sugar esters, polyol esters or polyol alkyl ethers,
complexing agents such as EDTA, NTA, β-alanine diacetic acid and phosphonic acids,
swelling and penetrating substances such as glycerol, propylene glycol monoethyl ether, carbonates, hydrogen carbonates, guanidines, ureas as well as primary, secondary and tertiary phosphates,
opacifiers such as latex, styrene/PVP and styrene/acrylamide copolymers,
pearlescent agents such as ethylene glycol mono- and distearate as well as PEG-3 distearate,
preservatives,
antioxidants.

With regard to further optional components and to the amounts of these components used, reference is expressly made to the relevant manuals known to the person skilled in the art.

The agents can be formulated in all conventional forms for styling agents, for example in the form of solutions, which can be applied to the hair as a lotion or pump or aerosol spray, or other preparations that are suitable for application on the hair.

The agents are preferably designed as a pump spray, aerosol spray, pump foam or aerosol foam. To this end the agents are presented in a dispensing device, which is either a compressed gas cylinder (aerosol container) additionally filled with a propellant or a non-aerosol container. Compressed gas cylinders, with the aid of which a product is distributed through a valve via the internal gas pressure in the cylinder, are by definition termed “aerosol containers”. In contrast to the aerosol definition, a “non-aerosol container” is defined as a container under normal pressure, with the aid of which a product is distributed through a pump system or squeezing system by means of a mechanical action.

The agents particularly preferably take the form of an aerosol hair foam or aerosol hair spray. The agent therefore preferably additionally contains at least one propellant.

Agents which take the form of an aerosol product can be produced in the usual way. All constituents of the agent, with the exception of the propellant, are generally introduced into a suitable pressure-resistant container. This is then closed with a valve. Finally the desired amount of propellant is added using conventional techniques.

Another embodiment provides a kit of parts comprising an aerosol dispensing device, in particular an aerosol spray device, and a composition containing, relative to the total weight of the composition,
about 30 to about 80 wt. %, preferably 40 to 70 wt. % and in particular about 50 to about 60 wt. % of an agent as described hereinabove; and
about 20 to about 70 wt. %, preferably about 25 to about 65 wt. % and in particular about 30 to about 60 wt. % of at least one propellant.

In the embodiment as an aerosol spray, propellants that are suitable are selected for example from N₂O, dimethyl ether, CO₂, air, alkanes having 3 to 5 carbon atoms, such as propane, n-butane, isobutane, n-pentane and isopentane, and mixtures thereof. Dimethyl ether, propane, n-butane, isobutane and mixtures thereof are preferred. According to a preferred embodiment the specified alkanes, mixtures of the specified alkanes or mixtures of the specified alkanes with dimethyl ether are used as the sole propellant. However, the invention expressly also comprises the incorporation of propellants of the chlorofluorocarbon type, but in particular fluorocarbons.

The propellant is preferably included in the agents of the aerosol spray embodiment in an amount from about 30 to about 60 wt. %, relative to the total weight of the agent.

Dimethyl ether or mixtures of propane and butane as the sole propellant are most particularly preferably used, in the weight ratio of propane to butane from about 20 to about 80 to about 15 to about 85. The mixtures are in turn preferably used in the agents in an amount from about 30 to about 55 wt. %, relative to the total weight of the agent. As used herein, butane is understood to mean n-butane, isobutane and mixtures of n-butane and isobutane. Dimethyl ether is most preferably used as the sole propellant.

In the given spray device the sizes of the aerosol droplets and the size distribution in each case can be adjusted by the ratio of propellant to the other constituents of the preparations.

The spray rate of the sprays is preferably about 6.5 to about 10.0 g/10 s.

Particularly preferred agents (aerosol sprays) are presented in an aerosol container having a stem valve with a stem bore of diameter about 0.27 to about 0.35 mm. Such valves are sold by Coster for example as KE or KEN valves.

In the embodiment as an aerosol foam, propellants that are suitable are selected for example from N₂O, dimethyl ether, CO₂, air, alkanes having 3 to 5 carbon atoms, such as propane, n-butane, isobutane, n-pentane and isopentane, and mixtures thereof. However, the invention expressly also comprises the incorporation of propellants of the chlorofluorocarbon type, but in particular fluorocarbons. According to the embodiment of an aerosol foam the specified alkanes, mixtures of the specified alkanes or mixtures of the specified alkanes with dimethyl ether are preferably used as the sole propellant. Particularly preferred propellants are dimethyl ether, propane, n-butane, isobutane and mixtures thereof.

In the given spray device the sizes of the foam bubbles and the size distribution in each case can be adjusted by the ratio of propellant to the other constituents of the preparations.

If conventional aerosol containers are used, aerosol foam products preferably contain the propellant in amounts from about 1 to about 35 wt. %, relative to the total weight of the aerosol foam product. Amounts from about 2 to about 30 wt. %, in particular from about 3 to about 15 wt. %, are particularly preferred.

Isopentane is preferably suitable as a propellant for expanding agents in gel form in a two-chamber aerosol container, it being incorporated into the agents and introduced into the first chamber of the two-chamber aerosol container. At least one further propellant that is different from isopentane and that builds a higher pressure in the two-chamber aerosol container than isopentane is introduced into the second chamber of the two-chamber aerosol container. The propellants of the second chamber are preferably selected from N₂O, dimethyl ether, CO₂, air, alkanes having 3 or 4 carbon atoms (such as propane, n-butane, isobutane) and mixtures thereof.

The use of the aforementioned additional preferred ingredients and of the amounts or ratios indicated as being preferred is naturally also preferred in the context of all aerosol foam embodiments.

The agents and products containing the agents, in particular aerosol hair sprays and aerosol hair foams, are characterized in particular in that they give the treated hair a very natural shine and strong hold. The invention secondly provides the use of an agent of the first subject matter of the invention for producing shine on keratin-containing fibers, in particular human hair.

The products containing these agents, in particular aerosol hair sprays, are characterized in particular in that they impart a very strong, lasting styling hold to the treated hair, even though the hair remains flexible. The invention therefore thirdly provides the use of an agent of the first subject matter of the invention for the temporary shaping of keratin-containing fibers, in particular human hair.

The invention also provides a method for treating keratin-containing fibers, in particular human hair, in which using a dispensing device an agent according to the first subject matter of the invention is applied to the keratin-am is applied to the kertta the kertat according to one of claims 1 to 11 isosol for is included as the cationic polymer. icontaining fibers as a spray.

It is preferable for the keratin-containing fibers to be shaped and for this shape to be fixed by the agent of the first subject matter of the invention.

After applying the agent it is also preferable for the agent to remain on the keratin-containing fibers, i.e. not to be rinsed out again. The aforementioned dispensing devices and aerosol products are preferred.

The invention also provides a method for treating keratin-containing fibers, in particular human hair, in which using a dispensing device an agent according to the first subject matter of the invention is expanded into a foam and the resulting foam is applied to the keratin-am is applied to the kertta the kertat according to one of claims 1 to 11 isosol for is included as the cationic polymer. icontaining fibers.

It is preferable for the keratin-containing fibers to be shaped and for this shape to be fixed by the agent of the first subject matter of the invention.

After applying the agent it is also preferable for the agent to remain on the keratin-containing fibers, i.e. not to be rinsed out again. The aforementioned dispensing devices are preferred.

### EXAMPLES

The following formulations were prepared by mixing the specified raw materials and were introduced into an aerosol can with a Coster KE valve and a Coster V06 standard micromist insert swirl nozzle. The aerosol cans were closed with the valve and then the corresponding propellant (dimethyl ether in this case) was added. Unless otherwise specified, the quantities given below are percentages by weight.

TABLE 1
Formulations

| Raw materials | E1 | E2 | E3 |
|---|---|---|---|
| 2-Amino-2-methylpropanol | 2.00 | 1.00 | 2.00 |
| Anionic strengthening polymer ¹ | 5.00 | 5.00 | 5.00 |
| Advantage LCA ² | 5.00 | 10.50 | 5.00 |
| Amphomer ® 28-4910 ³ | 2.00 | — | 2.00 |
| Dimethyl ether | 50.00 | 50.00 | 40.00 |

| | |
|---|---|
| Ethanol | <-----to 100-----> |

¹ Copolymer of styrene, methacrylic acid, 2-ethylhexyl acrylate and butyl acrylate (40% active substance in ethanol)
² Copolymer of N-vinylcaprolactam, N-vinylpyrrolidone and N,N-dimethylaminoethyl methacrylate (INCI name: Vinyl Caprolactam/VP/Dimethylaminoethylmethacrylate Copolymer, 37% active substance in ethanol) (Ashland)
³ INCI name: Octylacrylamide/Acrylates/Butylaminoethylmethacrylate Copolymer (100% active substance, powder) (Akzo Nobel)

After applying agents E1 to E3 to one hair strand in each case, these agents brought about an excellent styling hold. The hair was given a pronounced shine and felt soft.

1. An agent for treating keratin-containing fibers comprising a cosmetically acceptable carrier and
(a) at least one strengthening anionic copolymer, comprising at least one structural unit of formula (I) and at least one structural unit of formula (II) and at least one structural unit of formula (III) 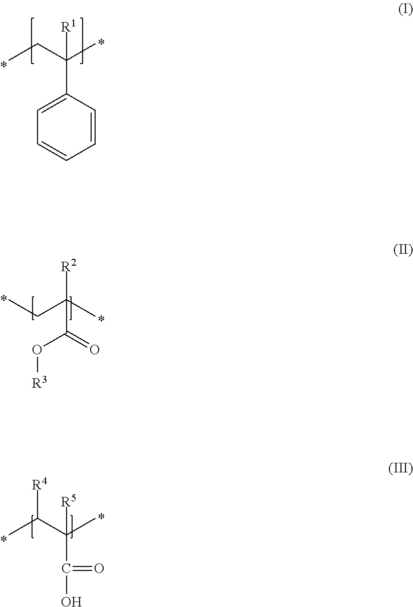
in which
R¹ denotes a hydrogen atom or a methyl group,
R² denotes a hydrogen atom or a methyl group,
R³ denotes a branched (C₁ to C₂₀) alkyl group, a branched (C₂ to C₆) hydroxyalkyl group or a *—(CH₂CH₂O)ₙ—R⁶ group where R⁶=branched (C₁ to C₂₀) alkyl and n=1 to 30, and
R⁴ and R⁵ each denote a hydrogen atom, or one of the two residues denotes a hydrogen atom and the other denotes a methyl group, and
(b) at least one polymer comprising at least one structural unit of formula (M1)

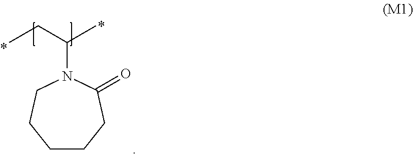

2. The agent according to claim 1, characterized in that the strengthening anionic copolymer comprises at least one structural unit of formula (I) and at least one structural unit of formula (IIa) and at least one structural unit of formula (IIb) and at least one structural unit of formula (III) 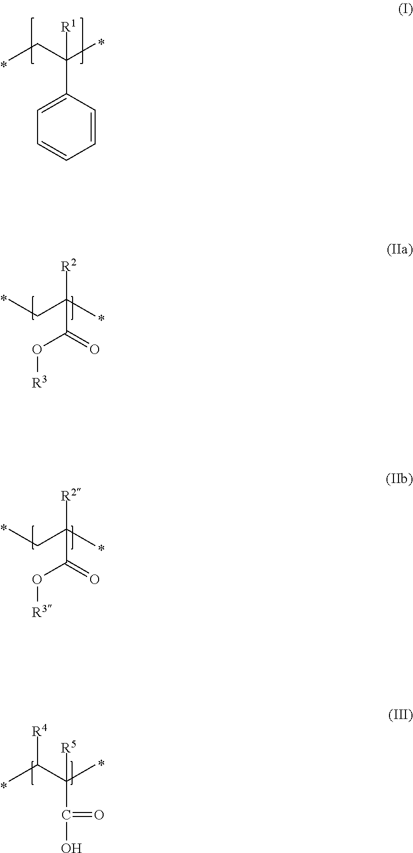
in which
R¹ denotes a hydrogen atom,
R² denotes a hydrogen atom,
R³ denotes an ethylhexyl group,
R²* denotes a hydrogen atom,
R³* denotes a butyl group,
R⁴ and R⁵ each denote a hydrogen atom, or one of the two residues denotes a hydrogen atom and the other denotes a methyl group.

3. The agent according to claim 1, characterized in that the strengthening anionic copolymer is included in an amount from about 0.01 to about 29.99 wt. %, relative to the total weight of the agent.

4. The agent according to claim 1, characterized in that the polymer of component (b) comprises at least one structural unit of formula (M1) and at least one structural unit of formula (M2) and at least one structural unit of formula (M3), 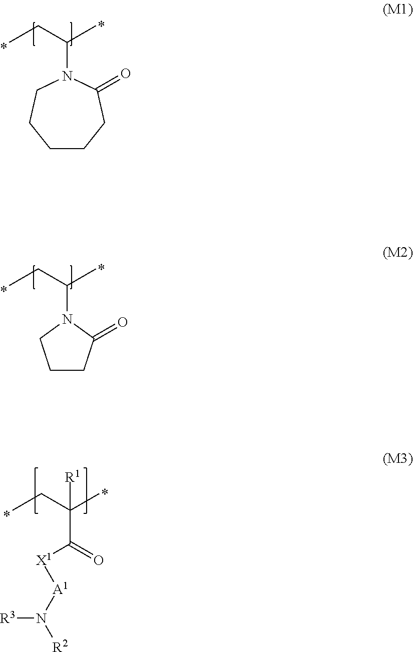
R¹ denotes a hydrogen atom or a methyl group,
X¹ denotes an oxygen atom or an NH group,
A¹ denotes an ethane-1,2-diyl, propane-1,3-diyl or butane-1,4-diyl group, and
R² and R³ independently of one another denote a (C₁ to C₄) alkyl group.

5. The agent according to claim 4, characterized in that in formula (M3) A¹ denotes an ethane-1,2-diyl group and X¹ denotes an oxygen atom.

6. The agent according to claim 4, characterized in that in formula (M3) A¹ denotes a propane-1,3-diyl group and X¹ denotes an NH group.

7. The agent according to claim 4, characterized in that in formula (M3) R² and R³ each denote a methyl group.

8. The agent according to claim 4, characterized in that in formula (M3) R¹ denotes a methyl group.

9. The agent according to claim 1, characterized in that the polymers of component (b) are included in an amount from about 0.05 to about 15.0 wt. %, relative to the total weight of the agent.

10. The agent according to claim 1, further comprising:
(c) at least one copolymer comprising at least one structural unit of formula (M5)

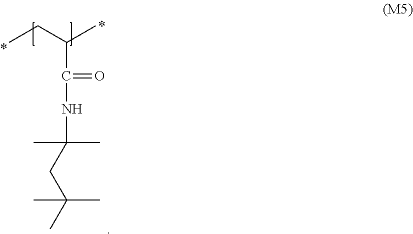

11. A method for treating keratin-containing fibers in which using a dispensing device an agent according to claim 1 is applied to the keratin-containing fibers as a spray.

12. (canceled)

12. The agent according to claim 1, wherein the keratin-containing fibers comprise human hair.

13. The method according to claim 10, wherein the keratin-containing fibers comprise human hair.

14. The agent according to claim 3, characterized in that the strengthening anionic copolymer is included in an amount from about 0.1 to about 14.9 wt. %, relative to the total weight of the agent.

15. The agent according to claim 3, characterized in that the strengthening anionic copolymer is included in an amount from about 0.1 to about 9.5 wt. %, relative to the total weight of the agent.

16. The agent according to claim 3, characterized in that the strengthening anionic copolymer is included in an amount from about 0.2 to about 7.0 wt. %, relative to the total weight of the agent.

17. The agent according to claim 9, characterized in that the polymers of component (b) are included in an amount from about 0.1 to about 7.0 wt. %, relative to the total weight of the agent.

18. The agent according to claim 2, characterized in that the strengthening anionic copolymer is included in an amount from about 0.01 to about 29.99 wt. %, relative to the total weight of the agent.

19. The agent according to claim 2, characterized in that the polymers of component (b) are included in an amount from about 0.05 to about 15.0 wt. %, relative to the total weight of the agent.

20. The agent according to claim 2, characterized in that the polymer of component (b) comprises at least one structural unit of formula (M1) and at least one structural unit of formula (M2) and at least one structural unit of formula (M3), 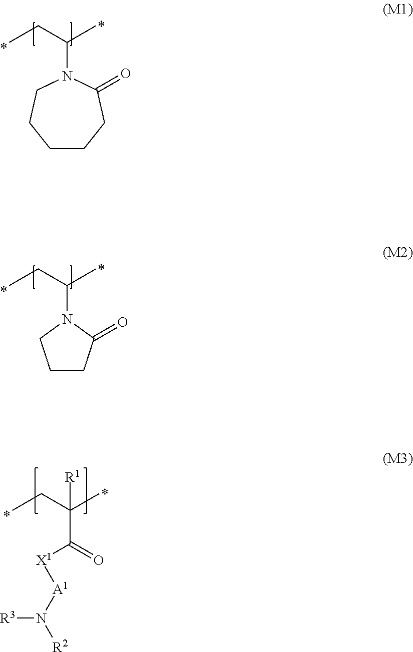
R¹ denotes a hydrogen atom or a methyl group,
X¹ denotes an oxygen atom or an NH group,
A¹ denotes an ethane-1,2-diyl, propane-1,3-diyl or butane-1,4-diyl group, and
R² and R³ independently of one another denote a (C₁ to C₄) alkyl group.
